(19) United States
(12) Patent Application Publication
(10) Pub. No.: US 2026/0277328 A1
(43) Pub. Date: Sep. 17, 2026

(54) TOUCH SCREEN INTERFACE MEMBRANE

(71) Applicant: GE Precision Healthcare LLC, Waukesha, WI (US)

(72) Inventors: Ramune Auzelyte, Milwaukee, WI (US); Steven M. Falk, Baltimore, MD (US); Saad Habayeb, Elm Grove, WI (US)

(73) Assignee: GE Precision Healthcare LLC, Waukesha, WI (US)

(21) Appl. No.: 19/075,988

(22) Filed: Mar. 11, 2025

(51) Int. Cl. G06F 3/01; A61B 34/00; G06F 3/041; G16H 40/63

(52) U.S. Cl. CPC G06F 3/016; G06F 3/0416; G16H 40/63; A61B 34/25

(57) ABSTRACT

A system includes a processor and a memory that includes instructions executable by the processor to receive a request for a touch screen user interface of a clinical medical application. Additionally, a reset signal is sent to a touch screen interface membrane. Further, a predetermined configuration for the touch screen interface membrane is selected. Additionally, the configuration is associated with the clinical medical software application and the touch screen user interface. Further, the configuration maps a touch screen control and a corresponding tactile effect. Additionally, a configuration signal is sent to the touch screen interface membrane that causes the tactile effect to be generated on a surface of the touch screen interface membrane in an area of the touch screen interface membrane that corresponds to a location of the touch screen control that is displayed on a display device having the touch screen interface membrane overlaid.

### BACKGROUND

The present disclosure generally relates to touch screen interfaces, and more particularly touch screen interface membranes.

In clinical applications, healthcare professionals may monitor the well-being of a patient while administering care. For example, an anesthesiologist may visually observe a patient during surgery while operating a device that administers anesthetic, oxygen, and other gases to the patient.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

A system includes a processor and a memory that includes instructions executable by the processor to receive a request for a touch screen user interface of a clinical medical application. Additionally, a reset signal is sent to a touch screen interface membrane. Further, a predetermined configuration for the touch screen interface membrane is selected. Additionally, the configuration is associated with the clinical medical software application and the touch screen user interface. Further, the configuration maps a touch screen control and a corresponding tactile effect. Additionally, a configuration signal is sent to the touch screen interface membrane that causes the tactile effect to be generated on a surface of the touch screen interface membrane in an area of the touch screen interface membrane that corresponds to a location of the touch screen control that is displayed on a display device having the touch screen interface membrane overlaid.

In some embodiments, the corresponding tactile effect is selected from a group consisting of a bump, a ridge, a groove, and a temperature differential between the area of the touch screen interface membrane and an area proximate to the area of the touch screen interface membrane.

In some embodiments, the system includes the display device and the touch screen interface membrane.

In some embodiments, the touch screen interface membrane includes excitation electrodes that are arranged in an array.

In some embodiments, the corresponding tactile effect includes a bump. Additionally, the touch screen interface membrane, in response to the configuration signal, applies a voltage between a pair of excitation electrodes that are disposed next to each other in the array.

In some embodiments, the corresponding tactile effect includes a groove. Additionally, the touch screen interface membrane, in response to the configuration signal, applies a plurality of voltages between a corresponding plurality of neighboring pairs of excitation electrodes. Further, the plurality of neighboring pairs of excitation electrodes is disposed in two neighboring columns of the array. Additionally, applying the voltage generates two ridges on the surface of the touch screen interface membrane. Further, the groove is disposed between the two ridges.

In some embodiments, the touch screen interface membrane includes an element selected from a group consisting of air and a viscous fluid.

In some embodiments, applying the voltage causes a portion of the element to flow to an area that is disposed proximate to the pair of excitation electrodes.

In some embodiments, the touch screen interface membrane, in response to the reset signal, deactivates the plurality of excitation electrodes.

A method includes receiving a request for a touch screen user interface of a clinical medical software application. Additionally, the method includes sending a reset signal to a touch screen interface membrane. Further, the method includes selecting a predetermined configuration for the touch screen interface membrane. Additionally, the predetermined configuration is associated with the clinical medical software application and the touch screen user interface. Further, the predetermined configuration includes a mapping between a touch screen control for the touch screen user interface and a corresponding tactile effect. Additionally, the method includes sending a configuration signal to the touch screen interface membrane that causes the touch screen interface membrane to generate the tactile effect on a surface of the touch screen interface membrane in an area of the touch screen interface membrane that corresponds to a location of the touch screen control that is displayed on a display device having the touch screen interface membrane overlaid.

In some embodiments, the corresponding tactile effect is selected from a group consisting of a bump, a ridge, a groove, and a temperature differential between the area of the touch screen interface membrane and an area proximate to the area of the touch screen interface membrane.

In some embodiments, the touch screen interface membrane includes excitation electrodes that are arranged in an array.

In some embodiments, the corresponding tactile effect includes a bump. Additionally, the touch screen interface membrane, in response to the configuration signal, applies a voltage between a pair of excitation electrodes that are disposed next to each other in the array.

In some embodiments, the corresponding tactile effect includes a groove. Additionally, the touch screen interface membrane, in response to the configuration signal, applies a plurality of voltages between a corresponding plurality of neighboring pairs of excitation electrodes. Further, the plurality of neighboring pairs of excitation electrodes is disposed in two neighboring columns of the array. Additionally, applying the voltage generates two ridges on the surface of the touch screen interface membrane. Further, the groove is disposed between the two ridges.

In some embodiments, the touch screen interface membrane includes an element selected from a group consisting of air and a viscous fluid.

In some embodiments, applying the voltage causes a portion of the element to flow to an area that is disposed proximate to the pair of excitation electrodes.

In some embodiments, the touch screen interface membrane, in response to the reset signal, deactivates the plurality of excitation electrodes.

A computer-readable storage medium includes instructions that are executable by a processor to receive a request for a touch screen user interface of a clinical medical application. Additionally, a reset signal is sent to a touch screen interface membrane having a plurality of excitation electrodes that are arranged in an array. Further, a predetermined configuration for the touch screen interface membrane is selected. Additionally, the configuration is associated with the clinical medical software application and the touch screen user interface. Further, the configuration maps a touch screen control and a corresponding tactile effect. Additionally, a configuration signal is sent to the touch screen interface membrane that causes the tactile effect to be generated on a surface of the touch screen interface membrane in an area of the touch screen interface membrane that corresponds to a location of the touch screen control that is displayed on a display device having the touch screen interface membrane overlaid.

In some embodiments, the corresponding tactile effect is selected from a group consisting of a bump, a ridge, a groove, and a temperature differential between the area of the touch screen interface membrane and an area proximate to the area of the touch screen interface membrane.

In some embodiments, the corresponding tactile effect includes a groove. Additionally, the touch screen interface membrane, in response to the configuration signal, applies a plurality of voltages between a corresponding plurality of neighboring pairs of excitation electrodes. Further, the plurality of neighboring pairs of excitation electrodes is disposed in two neighboring columns of the array. Additionally, applying the voltage generates two ridges on the surface of the touch screen interface membrane. Further, the groove is disposed between the two ridges.

Various other features, objects, and advantages of the invention will be made apparent from the following description taken together with the drawings.

### BRIEF DESCRIPTION OF THE DRAWINGS

The present disclosure is described with reference to the following Figures.

FIG. 1 is a diagram of an example system for a touch screen interface membrane, according to some embodiments of the present disclosure.

FIG. 2A is a diagram of a touch screen interface, according to some embodiments of the present disclosure.

FIG. 2B is a diagram of a touch screen interface membrane for a touch screen interface, according to some embodiments of the present disclosure.

FIG. 3A is a diagram of a touch screen interface, according to some embodiments of the present disclosure.

FIG. 3B is a diagram of a touch screen interface membrane for a touch screen interface, according to some embodiments of the present disclosure.

FIG. 4A is a diagram of a touch screen interface, according to some embodiments of the present disclosure.

FIG. 4B is a diagram of a touch screen interface membrane for a touch screen interface, according to some embodiments of the present disclosure.

FIG. 5 is side views of portions of example touch screen interface membranes, according to some embodiments of the present disclosure.

FIG. 6 is side views of portions of example touch screen interface membranes, touch panel, and liquid crystal display (LCD) panel, according to some embodiments of the present disclosure.

FIG. 7 is a top view of an example touch screen interface membrane, according to some embodiments of the present disclosure.

FIG. 8 is a flow chart of a method for a touch screen interface membrane, according to some embodiments of the present disclosure.

FIG. 9 is an exemplary a touch screen interface membrane manager, according to some embodiments of the present disclosure.

### DETAILED DESCRIPTION

In the present description, certain terms have been used for brevity, clarity and understanding. No unnecessary limitations are to be inferred therefrom beyond the requirement of the prior art because such terms are used for descriptive purposes only and are intended to be broadly construed.

As used herein, unless otherwise limited or defined, discussion of particular directions is provided by example only, with regard to particular embodiments or relevant illustrations. For example, discussion of “top,” “bottom,” “front,” “rear,” “left,” “right,” “horizontal,” “vertical,” and “longitudinal” features and/or relative motion, e.g., movement “up” and “down,” is generally intended as a description only of the orientation of such features relative to a reference frame of a particular example or illustration. Correspondingly, for example, a “top” feature may sometimes be disposed below a “bottom” feature (and so on), in some arrangements or embodiments. Additionally, or alternatively, embodiments may be arranged in a different orientation such that “top” and “bottom” features are arranged horizontally relative to each other, for example in a “left-to-right” orientation.

The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

The inventors have recognized problems with current techniques for administering care while monitoring a patient visually. For example, an anesthesiologist may use a touch screen device to administer gases to a patient during a surgery, such as anesthesia medications and oxygen. Additionally, the anesthesiologist may observe the patient visually to determine whether the patient wakes during a procedure, or experiences a reaction to the medication. As such, it may be useful for the anesthesiologist to administer the gases without looking away from the patient. Traditionally, care administration systems have included manual controls, dials, knobs, and other analog controls that a clinician or other operator might develop a muscle memory for operating without looking. However, with digital systems replacing traditional analog systems, a touch screen interface might now include the controls for operating medical devices. However, a touch screen interface is typically a flat screen surface that provides no tactile feedback from which a clinician or other operator might develop a muscle memory for operating without looking. Hence, the anesthesiologist may not be able to make specific selections on a touch screen interface for the gas administration without also looking at the touch screen, thus taking their eyes off the patient. More generally, there is currently no way for the clinician or other care device operator to operate care device controls using a touch screen interface while maintaining visual observation of a patient.

In view of the foregoing problems and challenges recognized by the inventors through their extensive research and experience in the field of patient monitoring and care administration, the inventors have developed the disclosed method and system for providing tactile and/or audio context to a care provider, such that the care provider can determine where on a touch screen to make specific selections without looking at the screen, thus administering care while maintaining visual observation of the patient. According to some embodiments of the present disclosure, a touch screen interface membrane may overlay a display of a touch screen device. Further, a system for managing the touch screen interface membrane may morph the membrane to configure bumps, ridges, and other elements that can provide tactile sensations that may help the clinician or other care device operator to develop the muscle memory for operating the care device. Thus, morphing the touch screen interface membrane to provide tactile elements in the locations of touch screen controls may provide indicators to the clinician or other care device operator with the muscle memory of a specific touch screen interface, which touch screen controls they are touching, and where to move their hands and/or fingers to access a specific touch screen control. Additionally, in some embodiments, this system may morph portions of the membrane to form bumps and/or indentations that the healthcare provider can press to select specific touch screen controls. Further, such embodiments may morph portions of the membrane into grooves that indicate a direction the healthcare provider may move their hand and/or finger to access a specific touch screen control. Additionally, in some embodiments, the touch screen interface membrane may incorporate temperature differences that indicate a specific touch screen control and/or a direction for moving to a specific touch screen control. Further, such embodiments may provide audio signals that indicate where on the touch screen interface membrane the healthcare provider's finger may be located. In these ways, such embodiments can improve patient monitoring and healthcare administration.

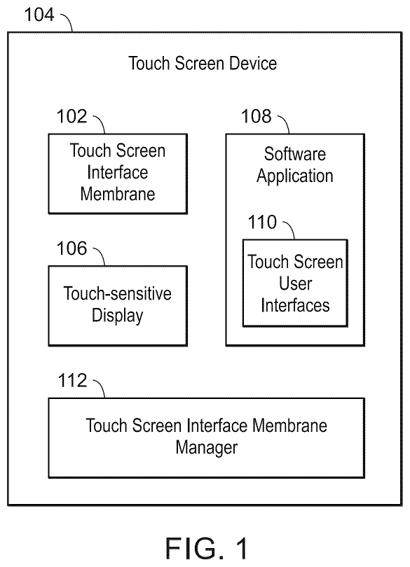

FIG. 1 is a diagram of an example system 100 for a touch screen interface membrane 102, according to some embodiments of the present disclosure. The system 100 includes a touch screen device 104 having a touch-sensitive display 106 overlaid with a touch screen interface membrane 102. The touch-sensitive display 106 may be a combination of a touch panel and a display panel. The display panel may be a liquid crystal display (LCD), organic light emitting diode (OLED) display, active-matrix OLED (AMOLED) display, plasma display, and the like. The touch panel may overlay the display panel. Further, the touch panel may be a thin, transparent layer of material covering the display panel. Additionally, the touch panel may be configured to react to a user touch with resistive, capacitive, infrared, acoustic wave, or like techniques in a manner that enables a driver (not shown) executing on the touch screen device 104 to determine the location, time, duration, and other parameters of the touch.

Further, the touch screen device 104 may execute a software application 108 that displays one or more touch screen user interfaces 110. According to some embodiments of the present disclosure, the software application may be a clinical medical software application, useful for administering care for, or monitoring physiological measures of, a patient in a clinical setting. The clinical setting may be a doctor's office, medical clinic, emergency room, operating room, hospital room, or like locations where a clinician or other healthcare provider may provide care for, and/or monitor a patient. As stated previously, a driver of the touch-sensitive display 106 may detect touch inputs on the touch-sensitive display's surface, and determine parameters describing the touch input. Further, the driver may provide these determined parameters to an operating system (not shown) of the touch screen device 104. For each touch input, the touch input parameters may include the touch's location, time of occurrence, duration, type, motion (e.g., swipe/slide, single tap, double tap), and the like. Further, the operating system may provide the touch input parameters to the software application 108 for processing. Accordingly, if the location of a touch input maps to a configured touch screen control on the touch screen user interface 110, the software application 108 may provide a response to the touch input based on the functionality associated with the configured touch screen control. A configured touch screen control may be a selection button, radio button, check box, data entry field, scroll bar, and the like.

According to some embodiments of the present disclosure, the touch screen interface membrane 102 may be a relatively thin electrical device that covers at least a portion of the touch-sensitive display 106. However, the touch screen interface membrane 102 may be transparent, translucent, or otherwise allow the touch screen user interface 110 to remain visible underneath. The touch screen interface membrane 102 may be secured to the touch-sensitive display 106 with adhesives, or some other securing element. Further, the touch-sensitive display 106 may be responsive to touch inputs on the touch screen interface membrane 102. Similar to the touch-sensitive display 106, the touch screen interface membrane may detect touch inputs on its surface, and provide parameters describing the touch input to an operating system.

According to some embodiments of the present disclosure, the touch screen interface membrane manager 112 may configure the surface of the touch screen interface membrane based on a predetermined configuration for the currently displayed touch screen user interface 110 of the software application 108. More specifically, the touch screen interface membrane 102 may configure its surface to provide tactile feedback to a user in regions of the touch screen interface membrane 102 that correspond to touch screen controls on the displayed touch screen user interface 110. In these ways, the touch screen interface membrane 102 may provide context to a user that is not looking at the touch screen device 104. By providing this tactile feedback, such embodiments may enable the user to determine where they can move their hand and/or finger to provide touch inputs to the software application 108. According to some embodiments, a clinician or other operator of the touch screen device 104 may use the touch screen interface membrane manager 112 to generate the predetermined configurations of one or more touch screen user interfaces 110. For example, a clinician or other operator may launch a software application 108, and select a touch screen user interface 110 for touch screen interface membrane configuration. Additionally, the clinician or other operator may launch the touch screen interface membrane manager 112 to generate a configuration for the selected touch screen user interface 110.

According to some embodiments of the present disclosure, the touch screen interface membrane 102 may configure its surface to generate raised areas (e.g., bumps) at locations that correspond to areas on the underlying touch screen user interface 110 with touch screen controls. Thus, when a user provides pressure at these bumps, the touch screen interface membrane 102 may come into contact with the touch-sensitive display 106, and provide an input to the software application 108. In this way, the user can provide touch inputs to the software application 108 through the touch screen interface membrane 102. Additionally, the touch screen interface membrane 102 may raise portions of the surface to create groove-like structures on its surface. Such grooves may provide guides to the user indicating directions to move their hand and/or fingers to provide and/or locate specific touch inputs. Additionally, the touch screen interface membrane 102 may configure its surface with areas of temperature differentials. In this way, the touch screen interface membrane 102 may provide context to the user indicating locations of touch screen controls, a direction to move their hand and/or fingers to locate touch screen controls, and the like. For example, a relatively cool area on the touch screen interface membrane 102 may indicate a location without touch input elements. Conversely, a relatively warm area may indicate an area corresponding to a touch input element and/or proximate to such an area. Additionally, the touch screen interface membrane 102 may use relatively warm and cool areas on its surface to delineate areas having separate touch input elements for the software applications. For example, neighboring touch input areas may alternate between relatively warmer and cooler surface temperatures. Alternatively, relatively cooler areas may indicate delineations between relatively warmer areas corresponding to touch input elements. Further, according to some embodiments of the present disclosure, the touch screen interface membrane 102 may provide combinations of raised areas, bumps, grooves, and temperature differentials to enable a user to provide touch inputs to the software application 108. Additionally, when the touch screen user interface 110 changes, (e.g., through the currently executing software application 108, or through the launch of a new software application 108), the touch screen interface membrane 102 may reset the touch screen interface membrane 102, select a configuration for the newly displayed touch screen user interface 110, and configure the touch screen interface membrane 102 based on the selected configuration.

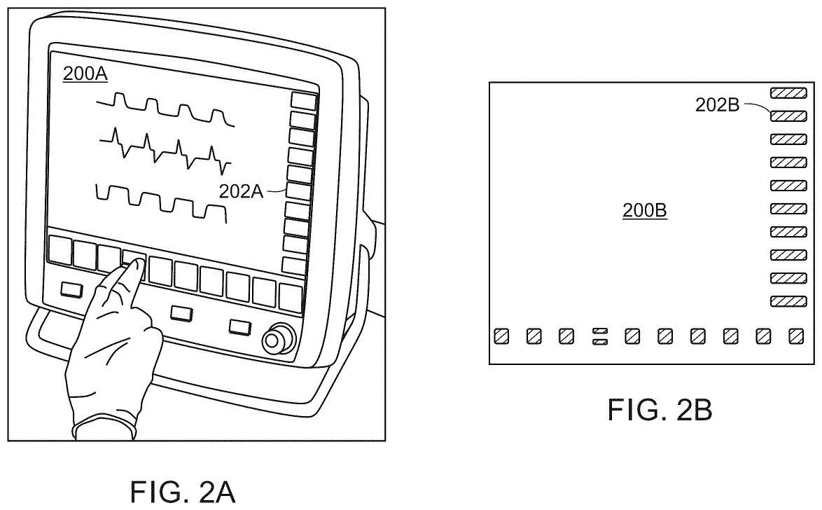

FIG. 2A is a diagram of an example touch screen user interface 200A, according to some embodiments of the present disclosure. The example touch screen user interface 200A represents a touch screen user interface for an anesthesia administration software application. The anesthesia administration software application may enable the clinician or other operator to administer anesthesia and other gases to a patient, and monitor physiological measures (e.g., respiration rate, heart rate) of the patient. In this example, the touch screen user interface 200A includes a display of waveforms 202 representing the physiological measures, and touch screen controls 204A for administering anesthesia and other gases. A software application, e.g., software application 108, may generate the touch screen user interface 200A, and respond to touch inputs on the touch screen controls 204A.

FIG. 2B is a diagram of example touch screen interface membrane 200B configured for the touch screen user interface 200A, according to some embodiments of the present disclosure. The example touch screen interface membrane 200B may overlay a touch-sensitive display surface that is showing the example touch screen user interface 200A. In this example, the configuration for the example touch screen interface membrane 200B includes control points 204B that correspond to the touch screen controls 204A. These control points may be raised surfaces and/or areas with temperature differential that a user could feel to determine the location of their hand and/or finger on the touch screen user interface 200A without looking at the touch screen user interface 200A.

As stated previously, the clinician or other operator may generate a touch screen interface membrane configuration for a touch screen user interface (e.g., example touch screen user interface 200A) using the touch screen interface membrane manager 112, described with respect to FIG. 1. More specifically, the clinician or other operator may launch the anesthesia administration software application, which may display the example touch screen user interface 200A. Additionally, the clinician or other operator may launch the touch screen interface membrane manager 112. Further, to generate the touch screen interface membrane 200B configuration, the clinician or other operator may tap each of the touch screen controls 204A of the example touch screen user interface 200A. The tap may indicate the selection of a bump, heat differential, or audio, configuration for the tapped location. Accordingly, the touch screen interface membrane manager 112 may use the touch input parameters provided by the operating system to identify the location of each tap. Further, the touch screen interface membrane manager 112 may generate a configuration that identifies the location of the tap, and indicates that the touch screen interface membrane configuration includes a raised surface at the location. Alternatively, the touch screen interface membrane manager 112 may indicate that the configuration includes a temperature differential and/or an audio signal at the location.

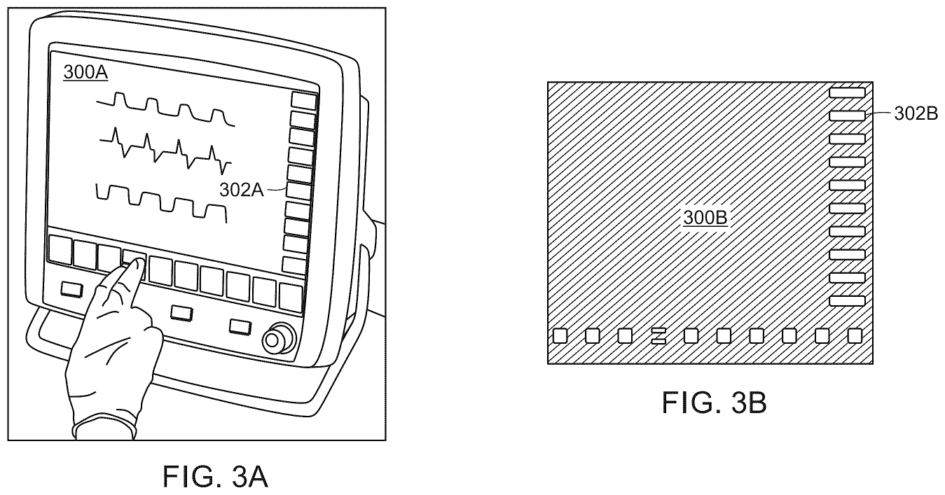

FIG. 3A is a diagram of a touch screen user interface 300A, according to some embodiments of the present disclosure. The touch screen user interface 300A may be similar to the touch screen user interface 200A.

FIG. 3B is a diagram of example touch screen interface membrane 300B for the touch screen interface, according to some embodiments of the present disclosure. In an alternative embodiment to the touch screen interface membrane 200B, the touch screen interface membrane 300B may be a transparent overlay of the user interface 300A, which includes cutouts 302B at the locations of the touch screen controls 302A. In this way, only touches to the cutout areas allow contact with the underlying touch-sensitive display surface, enabling the user to provide touch inputs to the software application.

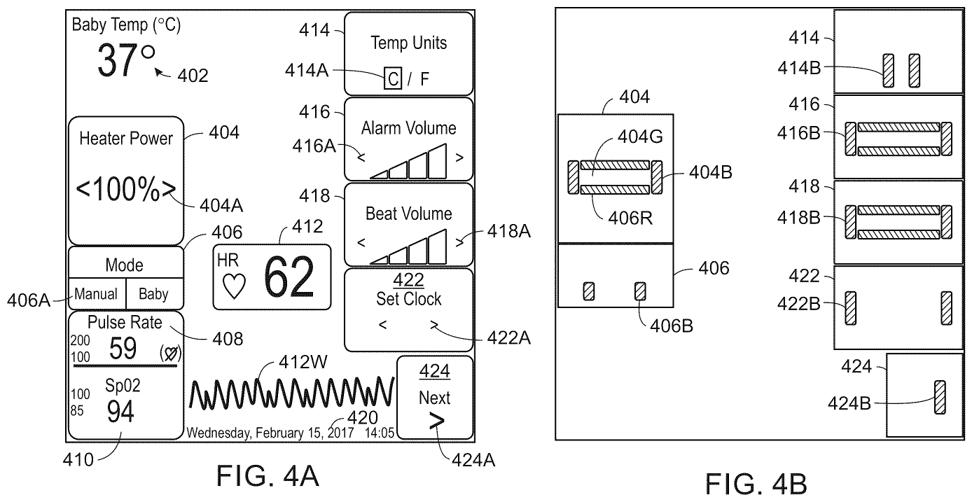

FIG. 4A is a diagram of an example touch screen user interface 400A, according to some embodiments of the present disclosure. A software application, such as an infant warmer application operating with an infant warmer device, may generate the touch screen user interface 400A. The touch screen user interface 400A may be configured to operate an infant warmer and display the vital statistics of an infant located in a bed of the infant warmer. In this example, the touch screen user interface 400A displays the baby's temperature 402, heater power level 404, mode 406, pulse rate 408, specific percentage of oxygen (SpO2) 410, heart rate 412, heart rate waveform 412W, temperature units 414, alarm volume 416, beat volume 418, date and time 420. The mode 406, “Manual,” or, “Baby,” may respectively indicate how the infant warmer application controls the radiant heater power. In Manual Mode, the clinician or other operator may control the radiant heater power with the touch screen user interface 400A of the infant warmer application, more specifically, a touch screen interface membrane overlaying the touch screen user interface 400A, described with respect to FIG. 4B. Alternatively, in Baby Mode, the infant warmer application may receive measurements from a temperature sensor attached to the infant. Hence, if the temperature measured by the sensor is below a threshold temperature, the infant warmer application may increase the radiant heater power. Conversely, if the infant's temperature is greater than the threshold temperature, the infant warmer application may decrease, or shut off, the radiant heater power. Further, the infant warmer application may receive, from other sensors attached to the infant, the baby's temperature 402, pulse 408, SpO2 410, and heart rate 412 for display on the interface 400A. Further, the infant warmer application may generate the heart rate waveform 412W based on the heart rate received over a predetermined time period. Additionally, the infant warmer application may initiate an alarm in certain circumstances, e.g., when the heart rate 412 and/or SpO2 410 detected by the sensors are below a certain threshold. Accordingly, the alarm volume 416 may indicate the current volume setting for the alarm. Additionally, the infant warmer application may emit a beep or similar sound that indicates each beat of the infant's heart. Accordingly, the beat volume 418 may indicate the current volume setting for that beep.

Further, the touch screen user interface 400A includes controls for configuring the infant warmer and monitoring functions. More specifically, the user interface 400A includes heat power selectors 404A, mode selector 406A, temperature unit selectors 414A, alarm volume selectors 416A, beat volume selectors 418A, clock setting selectors 422A, and “Next” selector 424. Touch inputs on the individual selectors may update settings of the infant warmer application. More specifically, a touch input on the left or right heat power selectors 404A may respectively lower or increase the heat power percentage. Additionally, a touch input on the “Manual,” or, “Baby,” mode selector 406A may change the mode for receiving vitals information, as described above. Further, a touch input on the, “C,” or, “F,” of the temperature unit selectors 414A may change the temperature units to Celsius or Fahrenheit, respectively. Additionally, a touch input on the left or right alarm volume selectors 416A may respectively lower or increase the alarm volume 416. Similarly, a touch input on the left or right beat volume selectors 418A may respectively lower or increase the beat volume 418. Further, a touch input on the left or right clock setting selectors 422A may respectively reverse or advance the time 420; and, if the time passes midnight in either direction, the date 420. In response, to a touch input of the “Next” selector 424, the infant warmer application may replace the touch screen user interface 400A with another touch screen interface, or potentially, launch another application, which may generate a new touch screen interface.

FIG. 4B is a diagram of an example touch screen interface membrane 400B configured for the touch screen user interface 400A, according to some embodiments of the present disclosure. In this configuration, the touch screen interface membrane 400B has control points for each of the selectors indicated in the touch screen user interface 400A. The control points may be configured as raised bumps on the touch screen interface membrane 400B. Accordingly, the touch screen interface membrane includes heat power control points 404B, mode control points 406B, temperature unit control points 414B, alarm volume control points 416B, beat volume control points 418B, clock setting control points 422B, and, “Next,” control point 424. Accordingly, a threshold amount of pressure applied on the touch screen interface membrane 400B at any of the control points may result in the touch screen interface membrane 400B coming into contact with the corresponding selector on the touch screen user interface 400A, and a corresponding response from the infant warmer application, as described above. Additionally, the touch screen interface membrane 400B is configured with a groove 404G, formed by two ridges 404R, for the heater power selector 404. The touch screen interface membrane 400B may generate the ridges by raising the surface in this areas in straight lines connecting to the ends of the heat power control points 404B. Accordingly, the groove 404G may provide a guide to the operator adjusting the heat power 404, along which they may slide their hand and/or finger between the control points for raising or lowering the heat power 404. While this example merely includes one groove for the heat power 404, some embodiments of the present disclosure may include grooves for other controls, such as the alarm volume 416 and beat volume. As stated previously, the touch screen interface membrane 400B may use temperature differentials alternatively, or in addition to, the raise control points described herein. Accordingly, instead of creating shapes on the surface at the control points indicated, in some embodiments, the touch screen interface membrane 400B may generate temperature differentials at the control points indicated. In other words, the control points may be areas that are warmer than the surrounding areas of the touch screen interface membrane 400B.

Further, in order to learn how to use the infant warmer application with the configuration shown for touch screen interface membrane 400B, without viewing the touch screen, a user may receive training before using this configuration in a clinical setting. Training may involve a training application for the configuration shown in the touch screen interface membrane 400B. For example, in response to a touch input from a control point on the touch screen interface membrane 400B, the training application may be configured to make an audio announcement describing the control selected. Hence, in response to pressure on the heat power control point 404B to increase heat power, the training application may use a speaker to pronounce, “Increase Heat.” Thus, through repetitive use in a training environment, a user may develop the muscle memory to operate the infant warmer application with the touch screen interface membrane 400B overlaid on the display for touch screen user interface 400A in a clinical setting.

To generate the configuration of the touch screen interface membrane 400B, the clinician or other operator may launch the infant warmer software application. The infant warmer software application may display the example touch screen user interface 400A. Alternatively, the clinician or other operator may select the example touch screen user interface 400A using selection options of the infant warmer software application. Additionally, the clinician or other operator may launch the touch screen interface membrane manager 112. Thus, to generate the configuration for the touch screen interface membrane 400B, the clinician or other operator may tap each of the touch screen controls 404A, 406A, 414A, 416A, 418A, 422A, 424A to indicate a bump configuration at each tapped location. Additionally, to indicate a ridge and groove configuration (e.g., groove 404G and ridges 406R), the clinician or other operator may perform a swipe/slide touch input at the location of the heater power percentage.

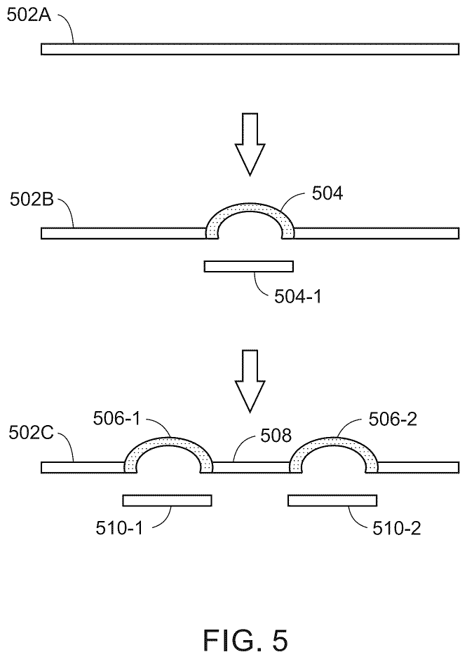

FIG. 5 is a diagram of portions of example touch screen interface membranes 502A, 502B, 502C, according to some embodiments of the present disclosure. The touch screen interface membrane 502A represents a flat, e.g., default, state, meaning that this touch screen interface membrane 502A is not configured to form any shapes, raised bumps, ridges, grooves, and the like. Further, the arrow pointing from the touch screen interface membrane 502A to touch screen interface membrane 502B indicates a transition to a new state. More specifically, the touch screen interface membrane 502B includes a bump 504, which the touch screen interface membrane may form over a control position 504-1 of a touch screen user interface. Applying at least a threshold pressure to the bump 504 may bring the touch screen interface membrane into contact with the control position 504-1. In response, the software application using the respective touch screen user interface may process the input accordingly. Further, the arrow pointing from the touch screen interface membrane 502B to touch screen interface membrane 502C indicates a transition to a new state. This transition may result from a user action, e.g., navigating through a menu tree underlying touch screen interface membrane 502B such that the underlying touch screen interface changes, and accordingly, the overlying touch screen interface membrane, i.e., touch screen interface membrane 502C. With respect to the new state, the touch screen interface membrane 502C may include bumps 506-1, 506-2. According to some embodiments of the present disclosure, the bumps 506-1, 506-2 may belong to ridges that form a groove 508 that provides tactile feedback to a user. In other words, the groove 508 may guide the user's hand and/or finger towards one or more control points. Alternatively, the bumps 506-1, 506-2 may represent control points. Accordingly, applying at least a threshold pressure to the bumps 506-1, 506-2 may bring the touch screen interface membrane into contact with control positions 510-1, 510-2, respectively. In response, the software application using the underlying touch screen user interface may process such inputs accordingly.

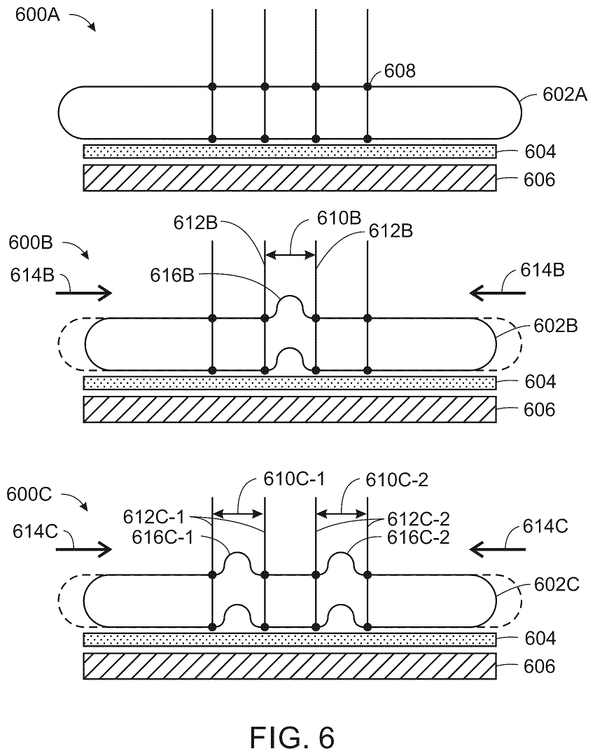

FIG. 6 is side views of portions 600A, 600B, 600C, of example touch screen interface membranes 602A, 602B, 602C, touch panel 604 and LCD panel 606, according to some embodiments of the present disclosure. The touch screen interface membranes 602A, 602B, 602C may be similar to the touch screen interface membranes 102, 200B, 400B, 502A, 502B, 502C, described with respect to FIGS. 1, 2B, 4B, and 5. The portions 600A, 600B, 600C respectively include touch screen interface membranes 602A, 602B, 602C overlaid on the touch panel 604, which is overlaid on the LCD panel 606. The LCD panel 606 may display a touch screen user interface (e.g., the touch screen user interface 110) generated by a software application (e.g., the software application 108). Additionally, the touch panel 604 may be a touch-sensitive device incorporated with the LCD panel 606 as part of the touch-sensitive display 106. Accordingly, an operating system of a touch screen device (e.g., touch screen device 104) may receive signals from the touch panel 604 when the touch screen interface membrane 602A, 602B, 602C, or another triggering element (e.g., a human finger) makes contact with the touch panel 604. Alternatively, the touch screen interface membrane 602A, 602B, 602C may include the touch panel elements. In other words, the touch screen interface membrane 602A, 602B, 602C may detect touch inputs on its surface, and provide the touch panel signals to the operating system of the touch screen device. In such embodiments, the touch screen interface membrane 602A, 602B, 602C, may overlay the LCD panel 606 without a touch panel 604 being disposed between.

According to some embodiments of the present disclosure, the touch screen interface membranes 602A, 602B, 602C, may include excitation electrodes 608 that are embedded into the touch screen interface membranes 602A, 602B, 602C. In this example, the touch screen interface membranes 602B, 602C morph in response to excitation (e.g., activation), or de-activation, of the embedded excitation electrodes 608. More specifically, the touch screen interface membranes 602A, 602B, 602C may contain air or a viscous fluid that may flow within the touch screen interface membranes 602A, 602B, 602C in response to voltage applied to the embedded excitation electrodes 608. For example, applying voltage across two neighboring excitation electrodes, may cause the air or viscous fluid to flow to the locations of the excitation electrodes 608 with the applied voltage, thus causing the area between the neighboring excitation electrodes 608 to bulge, and thus, morph, as indicated. In this way, according to some embodiments of the present disclosure, a touch screen interface membrane manager 112 may, or may not, activate, i.e., excite, the excitation electrodes 608 to generate the tactile feedback described herein. For example, the touch screen interface membrane 602A may represent a default, i.e., flat, state where the touch screen interface membrane manager 112 is not applying voltage to any of the excitation electrodes 608 of the touch screen interface membrane 602A. Accordingly, the excitation electrodes 608 of the touch screen interface membrane 602A are not excited (e.g., not activated). As such, the touch screen interface membrane 602A is similar to the touch screen interface membrane 502A, flat, i.e., without tactile feedback features that a human finger may sense as described herein.

The touch screen interface membrane 602B demonstrates a morphing of the surface in response to an excitation 610B. The excitation 610B is represented as an arrow pointing to two lines 612B, each of which is aligned with two excitation electrodes 608. According to some embodiments of the present disclosure, the touch screen interface membrane manager 112 may generate a voltage between the neighboring excitation electrodes 608 across each of the top and bottom surfaces of the touch screen interface membrane 602B between the indicated lines 612B. In this way, the touch screen interface membrane manager 112 may generate the excitation 610B. Further, the excitation 610B may cause air or viscous fluid within the touch screen interface membrane 602B to flow to the area between the excited, e.g., activated, excitation electrodes 608, thus causing the touch screen interface membrane 602B to generate a bump 616B on the top surface of the touch screen interface membrane 602B, generate a gap between the bottom surface of the touch screen interface membrane 602B and the touch panel 604, and contract at the edges. The arrows 614 indicate the contraction at the edges of the touch screen interface membrane 602B. Additionally, the dashed outlines of the touch screen interface membrane 602B represent the positions of the edges of the touch screen interface membrane 602B before the touch screen interface membrane manager 112 generates the excitation 610B. Accordingly, the solid outlines of the touch screen interface membrane 602B represent the positions of the edges of the touch screen interface membrane 602B after the touch screen interface membrane manager 112 generates the excitation 610B. In this way, the dashed and solid outlines, and the arrows 614B, indicate the resultant withdrawal of the edges of the touch screen interface membrane 602B towards the raised section of the touch screen interface membrane 602B, i.e., the bump 616B. Conversely, by de-activating the excitation electrodes 608 along the lines 612B, the air or viscous fluid within the touch screen interface membrane 602B between these excitation electrodes may flow away from the locations of the excitation electrodes 608, and return the touch screen interface membrane 602B to the state indicated by the touch screen interface membrane 602A.

However, while the touch screen interface membrane manager 112 is generating the excitation 610B, the bump 616B may represent a control point, meaning that applying at least a threshold pressure to the bump 616B may bring the bottom surface of the touch screen interface membrane 602B into contact with the touch panel 604. Hence, the touch panel 604 may signal the operating system of the touch screen device, indicating the location of the contact on the touch panel 604, and other parameters describing the type of contact (e.g., a single tap, double tap/touch, swipe/slide motion, and the like).

Similarly, the touch screen interface membrane 602C demonstrates a morphing of the touch screen interface membrane 602C in response to the excitations 610C-1, 610C-2. In this example, the excitation 610C-1 is represented as an arrow pointing to two lines 612C-1, each of which is aligned with two excitation electrodes 608. Similarly, the excitation 610C-2 is represented as an arrow pointing to two lines 612C-2, each of which is aligned with two excitation electrodes 608. According to some embodiments of the present disclosure, the touch screen interface membrane manager 112 may generate a voltage between the neighboring excitation electrodes 608 across each of the top and bottom surfaces of the touch screen interface membrane 602C between the indicated lines 612C-1. In this way, the touch screen interface membrane manager 112 may generate the excitation 610C-1. Similarly, the touch screen interface membrane manager 112 may generate a voltage between the neighboring excitation electrodes 608 across each of the top and bottom surfaces of the touch screen interface membrane 602C between the indicated lines 612C-2. In this way, the touch screen interface membrane manager 112 may generate the excitation 610C-2. Hence, the excitation 610C-1 may cause air or viscous fluid within the touch screen interface membrane 602C to flow between the excited, e.g., activated, excitation electrodes 608 at lines 612C-1, and between the activated excitation electrodes 608 at lines 612C-2, thus causing the touch screen interface membrane 602C to generate the bumps 616C-1, 616C-2 on the top surface of the touch screen interface membrane 602C, generate gaps between the bottom surface of the touch screen interface membrane 602C and the touch panel 604 and contract at the edges (as indicated by the arrows 614C, and the dashed outlines of the touch screen interface membrane 602C.

In some embodiments of the present disclosure, the bumps 612C-1, 612C-2 may represent control points, meaning that applying at least a threshold pressure to either (or both) of the bumps 612C may bring the bottom surface of the touch screen interface membrane 602C into contact with the touch panel 604. Hence, the touch panel 604 may signal the operating system of the touch screen device, indicating the location of the contact on the touch panel 604, and other parameters describing the type of contact (e.g., a single tap, double tap, slide motion, and the like).

Further, according to some embodiments of the present disclosure, the touch screen interface membrane manager 112 may generate excitations at consecutive lines of electrodes of the touch screen interface membrane 602C excited in this manner may generate ridges and a groove, similar to the ridges 404R and groove 404G, described with respect to FIG. 4B. In this way, the touch screen interface membrane 602C may provide tactile feedback that may be useful to guide a user's finger towards a control point on the touch screen interface membrane 602C.

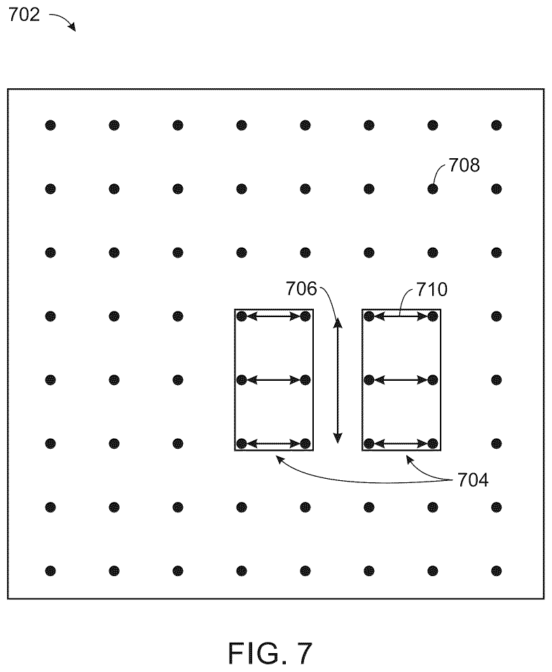

FIG. 7 is a top view of an example touch screen interface membrane 702, according to some embodiments of the present disclosure. The touch screen interface membrane 702 represents a touch screen interface membrane in a morphed state, having ridges 704 that form a groove 706 on its surface. In this example, the touch screen interface membrane manager 112 has generated excitations 710 between the indicated excitation electrodes 708. Accordingly, each column of excitations forms a ridge 704. Hence the neighboring ridges 704 form the groove 706 between. The ridges 704 may be similar to the ridges 406R, described with respect to FIG. 4B. Further, the groove 706 may be similar to the groove 406G. Accordingly, the groove 706 may represent an area that a clinician or other operator may use to perform a swipe/slide action on the touch screen interface membrane. Additionally, or alternatively, the groove 706 may represent another example of tactile feedback that the clinician or other operator may use to determine their finger's location on the touch screen interface membrane 702 when they are not looking at the touch screen interface membrane 702.

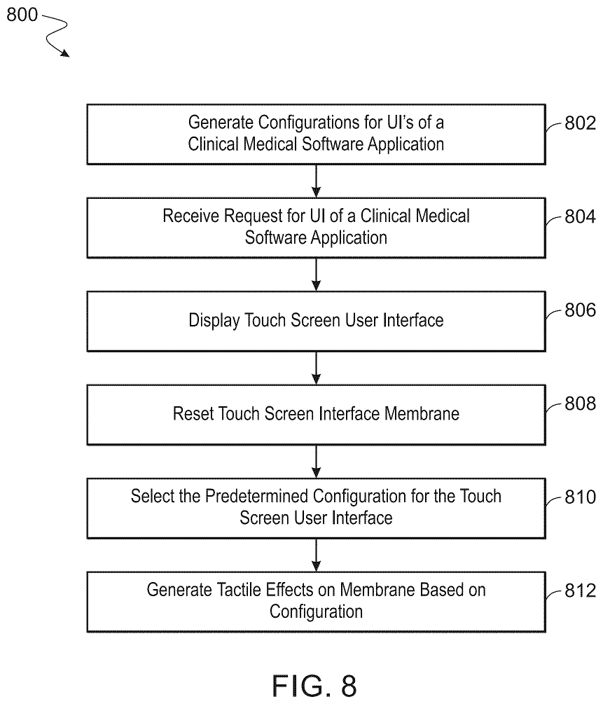

FIG. 8 is a flow chart of a method 800 for a touch screen interface membrane 102, according to some embodiments of the present disclosure. A touch screen interface membrane manager 112 and software application 108, e.g., software application may perform the method 800.

At operation 802, the touch screen interface membrane manager 112 may generate touch screen interface membrane configurations for the user interfaces (UT's) of a clinical medical software application. As stated previously, the clinician or other operator may launch the software application 108 having the touch screen user interface 110 for which the touch screen interface membrane 102 is to be configured. Additionally, the clinician or other operator may select the touch screen user interface 110 by navigating the touch screen user interfaces 110 of the software application 108 until the touch screen user interface 110 is displayed. Additionally, the clinician or other operator may launch the touch screen interface membrane manager 112, which may generate the configuration for the selected touch screen user interface 110 based on tap, swipe/slide, and the like, touch inputs that the clinician or other operator provides, as described with respect to FIGS. 1, 2B, and 4B.

At operation 804, the touch screen interface membrane manager 112 may receive a request for a user interface of a clinical medical software application. The software application 108 may generate the request when the software application launches, or when the software application is navigating to a new touch screen user interface 110.

At operation 806, the software application 108 may display the touch screen user interface 110. The touch screen user interface 110 may be a home or default interface that the software application 108 displays upon launch. Alternatively, the touch screen user interface 110 may be a different touch screen user interface 110 to which the clinician or other operator navigates.

At operation 808, the touch screen user interface membrane manager 112 may reset the touch screen interface membrane 102. Resetting the touch screen interface membrane 102 may involve stopping any active excitations. In other words, the touch screen interface membrane manager 112 may reset the touch screen interface membrane 102 by stopping any applied voltages across the touch screen interface membrane's excitation electrodes.

At operation 810, the touch screen user interface membrane manager 112 may select the predetermined configuration for the displayed touch screen user interface 110. Selecting the predetermined configuration may be based on the software application 108 and touch screen user interface 110.

At operation 812, the touch screen user interface membrane manager 112 may generate tactile effects on the touch screen interface membrane 102 based on the predetermined configuration. As stated previously, the predetermined configuration may include bumps, ridges, grooves, temperature differentials, and the like. To generate bumps, ridges, grooves, and/or other raised and indented surfaces, the touch screen interface membrane manager 112 may apply voltage across excitation electrodes at the locations indicated by the predetermined configuration.

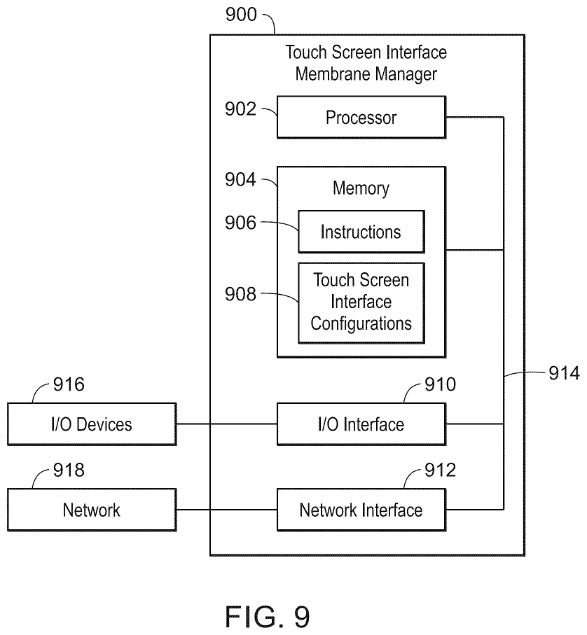

FIG. 9 is an exemplary touch screen interface membrane manager 900, according to some embodiments of the present disclosure. The example touch screen interface membrane manager 900 may configure the surface of a touch screen interface membrane 102 to provide tactile feedback to a user in regions of the touch screen interface membrane that correspond to touch screen controls on a displayed touch screen user interface, as described with respect to FIGS. 1, 2A, 2B, 3A, 3B, 4A, 4B, and 5-8. In this example, the touch screen interface membrane manager 900 includes a processor 902, memory 904, input-output (I/O) interface 910, and network interface 912, which may be connected by an interconnect 914. The processor 902 may be a computer processing circuit (e.g., a central processing unit (CPU)) that retrieves and executes programming instructions 906 stored in the memory 904 to perform the functionality described herein. The interconnect 914 may move data, such as programming instructions, between the processor 902, memory 904, I/O interface 910, and network interface 912. The interconnect 914 may include one or more buses.

The memory 904 may be a computer memory or storage device, including volatile memory, such as a random access memory (RAM) device (e.g., static RAM, dynamic RAM, and the like), non-volatile memory, such as a hard disk drive, solid state device (SSD), removable memory cards, optical storage, flash memory devices, and the like. In some examples, the memory 904 may include volatile and non-volatile memory devices. Further, the memory 904 may store instructions 906, and touch screen interface configurations 908. The touch screen interface configurations 908 may be similar to the touch screen interface configurations, described with respect to FIGS. 2B and 4B.

Additionally, the touch screen interface membrane manager 900 may be in electronic communication with I/O devices 916 through the I/O interface 910, and with a network 918 through the network interface 912. The I/O devices 916 may capture inputs and provide outputs as described herein. The network 918 may be an electronic communication network, such as a local area network, wide area network, and the like, for processing communications between the touch screen interface membrane manager 900 and the machine learning models and AI software products described herein. In some examples, the network 918 may be wired, wireless (e.g., wi-fi, Bluetooth, or cellular), or some other computer communication network.

In some embodiments, the touch screen interface membrane manager 900 may be a server computer or similar device without a user interface but which receives requests from other computer systems having one or more user interfaces. Further, in some embodiments, the touch screen interface membrane manager 900 may be a portable computer, laptop, tablet computer, pocket computer, telephone, smart phone, or the like.

In some embodiments of the present disclosure, the operations of the methods described herein may occur out of the order noted in the Figures. For example, two opeations shown in succession may, alternatively, be executed as one operation, executed concurrently, substantially concurrently, in a partially or wholly temporally overlapping manner, or in the reverse order, depending upon the functionality involved.

As used herein, the term, mechanism, can encompass hardware, software, firmware, or any suitable combination thereof. In some embodiments, any suitable computer readable media can be used for storing instructions for performing functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A system, comprising:
a processor; and
a memory comprising instructions executable by the processor to:
receive a request for a touch screen user interface of a clinical medical software application;
send a reset signal to a touch screen interface membrane;
select a predetermined configuration for the touch screen interface membrane, the predetermined configuration:
being associated with the clinical medical software application and the touch screen user interface; and
comprising a mapping between a touch screen control for the touch screen user interface and a corresponding tactile effect; and
send a configuration signal to the touch screen interface membrane that causes the touch screen interface membrane to generate the corresponding tactile effect on a surface of the touch screen interface membrane in an area of the touch screen interface membrane, wherein pressure applied on the corresponding tactile effect causes the touch screen interface membrane to make contact with a touch panel of a touch screen device, wherein the area of the touch screen interface membrane corresponds to a location where the touch screen control is displayed on a display layer of the touch screen device having the touch screen interface membrane overlaid.

2. The system of claim 1, wherein the corresponding tactile effect is selected from a group consisting of a bump, a ridge, a groove, and a temperature differential between the area of the touch screen interface membrane and an area proximate to the area of the touch screen interface membrane.

3. (canceled)

4. The system of claim 1, wherein the touch screen interface membrane comprises a plurality of excitation electrodes that are arranged in an array.

5. The system of claim 4, wherein the corresponding tactile effect comprises a bump, and wherein the touch screen interface membrane, in response to the configuration signal, applies a voltage between a pair of excitation electrodes that are disposed next to each other in the array.

6. The system of claim 4, wherein the corresponding tactile effect comprises a groove, and wherein the touch screen interface membrane, in response to the configuration signal, applies a plurality of voltages between a corresponding plurality of neighboring pairs of excitation electrodes, wherein the plurality of neighboring pairs of excitation electrodes is disposed in two neighboring columns of the array, and wherein applying the voltage generates two ridges on the surface of the touch screen interface membrane, wherein the groove is disposed between the two ridges.

7. The system of claim 5, wherein the touch screen interface membrane comprises an element selected from a group consisting of air and a viscous fluid.

8. The system of claim 7, wherein applying the voltage causes a portion of the element to flow to an area that is disposed proximate to the pair of excitation electrodes.

9. The system of claim 4, wherein the touch screen interface membrane, in response to the reset signal, deactivates the plurality of excitation electrodes.

10. A method, comprising:
receiving a request for a touch screen user interface of a clinical medical software application;
sending a reset signal to a touch screen interface membrane;
selecting a predetermined configuration for the touch screen interface membrane, the predetermined configuration:
being associated with the clinical medical software application and the touch screen user interface; and
comprising a mapping between a touch screen control for the touch screen user interface and a corresponding tactile effect; and
send a configuration signal to the touch screen interface membrane that causes the touch screen interface membrane to generate the corresponding tactile effect on a surface of the touch screen interface membrane in an area of the touch screen interface membrane, wherein pressure applied on the corresponding tactile effect causes the touch screen interface membrane to make contact with a touch panel of a touch screen device, wherein the area of the touch screen interface membrane corresponds to a location where the touch screen control is displayed on a display layer of the touch screen device having the touch screen interface membrane overlaid.

11. The method of claim 10, wherein the corresponding tactile effect is selected from a group consisting of a bump, a ridge, a groove, and a temperature differential between the area of the touch screen interface membrane and an area proximate to the area of the touch screen interface membrane.

12. The method of claim 10, wherein the touch screen interface membrane comprises a plurality of excitation electrodes that are arranged in an array.

13. The method of claim 11, wherein the corresponding tactile effect comprises a bump, and wherein the touch screen interface membrane, in response to the configuration signal, applies a voltage between a pair of excitation electrodes that are disposed next to each other in the array.

14. The method of claim 11, wherein the corresponding tactile effect comprises a groove, and wherein the touch screen interface membrane, in response to the configuration signal, applies a plurality of voltages between a corresponding plurality of neighboring pairs of excitation electrodes, wherein the plurality of neighboring pairs of excitation electrodes is disposed in two neighboring columns of the array, and wherein applying the voltage generates two ridges on the surface of the touch screen interface membrane, wherein the groove is disposed between the two ridges.

15. The method of claim 14, wherein the touch screen interface membrane comprises an element selected from a group consisting of air and a viscous fluid.

16. The method of claim 15, wherein applying the voltage causes a portion of the element to flow to an area that is disposed proximate to the pair of excitation electrodes.

17. The method of claim 12, wherein the touch screen interface membrane, in response to the reset signal, deactivates the plurality of excitation electrodes.

18. A non-transitory computer-readable storage medium comprising instructions that are executable by a processor to:
receive a request for a touch screen user interface of a clinical medical software application;
send a reset signal to a touch screen interface membrane comprising a plurality of excitation electrodes that are arranged in an array;
select a predetermined configuration for the touch screen interface membrane, the predetermined configuration:
being associated with the clinical medical software application and the touch screen user interface; and
comprising a mapping between a touch screen control for the touch screen user interface and a corresponding tactile effect; and
send a configuration signal to the touch screen interface membrane that causes the touch screen interface membrane to generate the corresponding tactile effect on a surface of the touch screen interface membrane in an area of the touch screen interface membrane, wherein pressure applied on the corresponding tactile effect causes the touch screen interface membrane to make contact with a touch panel of a touch screen device, wherein the area of the touch screen interface membrane corresponds to a location where the touch screen control is displayed on a display layer of the touch screen device having the touch screen interface membrane overlaid.

19. The non-transitory computer-readable storage medium of claim 18, wherein the corresponding tactile effect is selected from a group consisting of a bump, a ridge, a groove, and a temperature differential between the area of the touch screen interface membrane and an area proximate to the area of the touch screen interface membrane.

20. The non-transitory computer-readable storage medium of claim 18, wherein the corresponding tactile effect comprises a groove, and wherein the touch screen interface membrane, in response to the configuration signal, applies a plurality of voltages between a corresponding plurality of neighboring pairs of excitation electrodes, wherein the plurality of neighboring pairs of excitation electrodes is disposed in two neighboring columns of the array, and wherein applying the voltage generates two ridges on the surface of the touch screen interface membrane, wherein the groove is disposed between the two ridges.
